(19) United States
(12) Patent Application Publication
(10) Pub. No.: US 2014/0257041 A1
(43) Pub. Date: Sep. 11, 2014

(54) PERINEAL RETRACTOR

(71) Applicant: University of Rochester, Rochester, NY (US)

(72) Inventor: Granville Lloyd, Madison, WI (US)

(73) Assignee: University of Rochester, Rochester, NY (US)

(21) Appl. No.: 14/200,862

(22) Filed: Mar. 7, 2014

Related U.S. Application Data
(60) Provisional application No. 61/774,697, filed on Mar. 8, 2013.

(51) Int. Cl. A61B 17/02; A61B 19/00

(52) U.S. Cl. CPC A61B 17/02; A61B 19/38; A61B 2017/0212
USPC 600/206; 600/226; 600/235

(57) ABSTRACT

An apparatus for perineal retraction during a prostatectomy includes a handle portion with a first end, a second end and a curvilinear segment between the first and second end. The second end faces in a direction toward the first end. The apparatus also includes a head portion including a first surface on one side of the head portion and a second surface on an opposite side of the head portion, the second surface being connected to or engageable with the second end of the handle. Use of the apparatus for perineal retraction during prostatectomy is also described.

This application claims the priority benefit of U.S. Provisional Patent Application Ser. No. 61/774,697, filed Mar. 8, 2013, which is hereby incorporated by reference in its entirety.

### FIELD OF THE INVENTION

This invention relates to a perineal retractor, optionally having portions thereof integrated into surgical drapery, and its use during a prostatectomy.

### BACKGROUND OF THE INVENTION

Many surgeons use direct perineal pressure during open prostatectomy. Since the advent of robotically assisted prostatectomy in 2000, this type of minimally invasive surgery has become widespread, with now over 80% of the roughly 90,000 radical prostatectomies annually performed in the United States being conducted in this fashion. The great utility of perineal pressure remains and while the patient is positioned well to allow for application of perineal pressure, the location of a robotic surgical machine directly between the legs of the patient makes this difficult to achieve. Routinely, an assistant must reach in an awkward fashion between the arms of the machine and attempt blindly to find the best spot to apply perineal pressure to the patient, while in a semi-standing position, and all the while still performing the other tasks of assistance during this part of the surgical procedure.

The present invention is a simple and cost-effective solution to the problem of applying pressure to the perineum during an open prostatectomy or anastomotic portion of robotic prostatectomy surgery, and therefore overcomes these deficiencies in the art.

### SUMMARY OF THE INVENTION

One aspect of the present invention relates to an apparatus, namely a perineal retractor, for use during a prostatectomy. The apparatus includes a handle portion with a first end, a second end, and a curvilinear segment between the first and second ends. The second end faces in a direction toward the first end. The apparatus also includes a head portion including a first surface on one side of the head portion and a second surface on an opposite side of the head portion, the second surface being connected to or engageable with the second end of the handle portion. The head portion is sized and configured to allow a user to apply pressure to the perineum during prostatectomy procedures.

A second aspect of the present invention relates to an apparatus for perineal retraction during a prostatectomy. The apparatus includes a head portion including a first surface on one side of the head portion and a second surface on an opposite side of the head portion, the head portion being sized and configured for placement against a perineum. The apparatus also includes a pad covering a substantial portion of the first surface of the head portion; and a surgical drapery layer secured either between the first surface of the head portion and the pad or to the second surface of the head portion.

A third aspect of the present invention relates to an apparatus for perineal retraction during a prostatectomy. The apparatus includes a head portion including a first surface on one side of the head portion and a second surface on an opposite side of the head portion, the head portion being sized and configured for placement with the first surface adjacent a perineum during prostatectomy procedures. The apparatus also includes a surgical drapery layer secured to either the first surface or the second surface of the head portion.

A fourth aspect of the invention relates to a method of performing a prostatectomy that includes applying against the perineum of a patient the head portion of an apparatus according to the first, second, or third aspect of the invention, and retracting the perineum upon manipulation of the head portion. Manipulation of the head portion can be achieved manually, by hand, or through the use of a handle portion, if desired.

The present invention achieves an important advance for performing prostatectomy surgery, particularly the often-used robotically assisted prostatectomy surgery. Given the proximity of the rectum to the perineal region, which is where pressure needs to be applied, this makes the preservation of a sterile environment difficult. The desire to minimize direct, manual manipulation of the region is facilitated greatly by use of the inventive retractor, regardless of whether it is integrated with surgical drapery.

### BRIEF DESCRIPTION OF THE DRAWINGS

FIG. 1 is a side elevational view of a first embodiment of an apparatus for perineal retraction during a prostatectomy;

FIG. 2 illustrates a head portion of the apparatus, along line 2-2 in FIG. 1;

FIG. 3 illustrates a second embodiment of an apparatus for perineal retraction during a prostatectomy;

FIG. 4 is an enlarged, side elevational view of the head portion of the second embodiment of an apparatus, with the retaining member on the back side of the head portion partially broken away to illustrate the pivotal ball-and-socket connection between the handle portion and the head portion;

FIG. 5 illustrates a head portion of a third embodiment of an apparatus an apparatus for perineal retraction during a prostatectomy, here shown with its handle portion broken away;

FIG. 6 illustrates a head portion of a fourth embodiment of an apparatus an apparatus for perineal retraction during a prostatectomy, here shown with its handle portion broken away;

FIG. 7 illustrates a head portion of a fifth embodiment of an apparatus for perineal retraction during a prostatectomy, here shown integrated with a surgical drapery and with its handle portion broken away;

FIG. 8 illustrates a head portion of a sixth embodiment of an apparatus for perineal retraction during a prostatectomy, here shown integrated with a surgical drapery and with its handle portion broken away; and

FIG. 9 is a side elevational view of a seventh embodiment of an apparatus for perineal retraction during a prostatectomy.

### DETAILED DESCRIPTION OF THE INVENTION

The present invention relates to a perineal retractor for use during a prostatectomy. Because of its shape and utility, the apparatus allows an assistant positioned near a patient's torso (or, in some embodiments, adjacent the robotic device) to apply appropriate pressure to the perineum during an open prostatectomy or the anastamotic phase of robotic prostatectomy.

As described in more detail below, the several embodiments of the present invention each include a handle portion and a head portion, which during use is intended to contact the patient's perineum. The handle portion includes a first end with a grip region, a second end, and a curvilinear segment between the first and second ends. The curvilinear segment in certain embodiments has a gooseneck configuration, thereby positioning the head portion so that it faces the first end (grip region) of the handle portion. The head portion is, importantly, sized and configured to allow for the application of pressure to the perineum during the prostatectomy. In this regard, it is contemplated that the head portion can be provided in multiple sizes and shapes (i.e., configurations) to allow the apparatus to be utilized with patients of different size and body type. In the several embodiments, the head portion has a variety of constructions that connect it or allow it to be engaged by the handle portion while under control of a user. In one approach, the head portion can be rigidly connected to the second end of the handle portion to maintain it a fixed position. In another approach, the head portion can be pivotally connected to the second end of the handle portion to allow angular adjustment of the positional relationship between the handle portion and head portion. In yet another approach, the head portion may be freely engageable or disengageable from the handle portion to facilitate the use of various types of head portions, including a disposable head portion. In yet another approach, the head portion may be integrated into surgical drapery.

In the several embodiments describe herein, the handle portion and the head portion can be reusable, in which case they are preferably made of surgical grade stainless steel, titanium, aluminum or any other durable material suitable for repeat usage, particularly metal materials that are free of impurities and appropriate for surgical procedures. The apparatus can therefore be reutilized after appropriate sterilization.

Alternatively, both the handle portion and the head portion can be disposable. Where disposability of all or part of the apparatus 10 is desired, a rigid biocompatible plastic such as an acrylonitrile butadiene styrene (“ABS”) plastic, or a thermoplastic material such as a polyester, polypropylene, polyethylene, or other plastic material that provides lightweight and durability can be used.

In an alternative approach, the handle portion can be reusable and the head portion can be disposable, in which case the above-described materials can be selected for these portions, as appropriate.

Regardless of whether disposable or re-usable materials are utilized in the manufacture of the retractor portions, these portions can be prepared in their desired shape and configuration by, e.g., die-casting, stamping, or injection molding, as is well known in the art. Other procedures for their manufacture can also be used. Where coupling of the head portion to the handle portion is required for producing the assembled product, such assembly can be carried out during production or by the end-user, as is appropriate.

As discussed herein, the head portion may include a layer of padding applied to the face that is intended to contact the patient. Suitable padding materials include, without limitation, open-cell-structured foams also known as reticulated foams, or gel materials, or any other suitable material. Any adhesive materials used to secure certain components during use can be adhesive materials that are intended to be permanent connections, e.g., padding applied to a disposable head, or releasable adhesive connections, e.g., padding applied to a reusable head.

Of course, the foregoing identification of materials is exemplary only, and numerous variations, substitutions, and changes in material can be made by those of ordinary skill in the art without departing from the scope of the present invention.

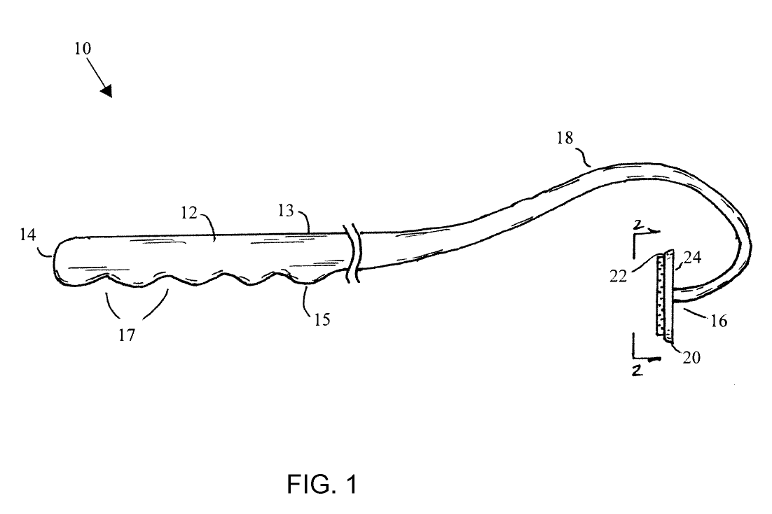

FIG. 1 illustrates a first embodiment of the apparatus 10. This embodiment includes a handle portion 12 with a first end 14, a second end 16, and a curvilinear segment 18 between the first and second end. The curvilinear segment 18 has a gooseneck configuration such that the second end 16 faces in a direction toward the first end 14. The apparatus 10 also includes a head portion 20 including a first surface 22 on one side of the head portion 20 and a second surface 24 on an opposite side of the head portion 20. In this embodiment, the second surface 24 is integrally connected to the second end 16 of the apparatus 10. The handle portion 12 further includes an upper side 13 and a lower side 15 opposite the upper side 13, and a plurality of spaced indentations 17 disposed in the lower side 15 of the handle portion 12. This construction allows a user to grip and manipulate the apparatus 10 with a single hand while performing other tasks during the surgery with their free hand.

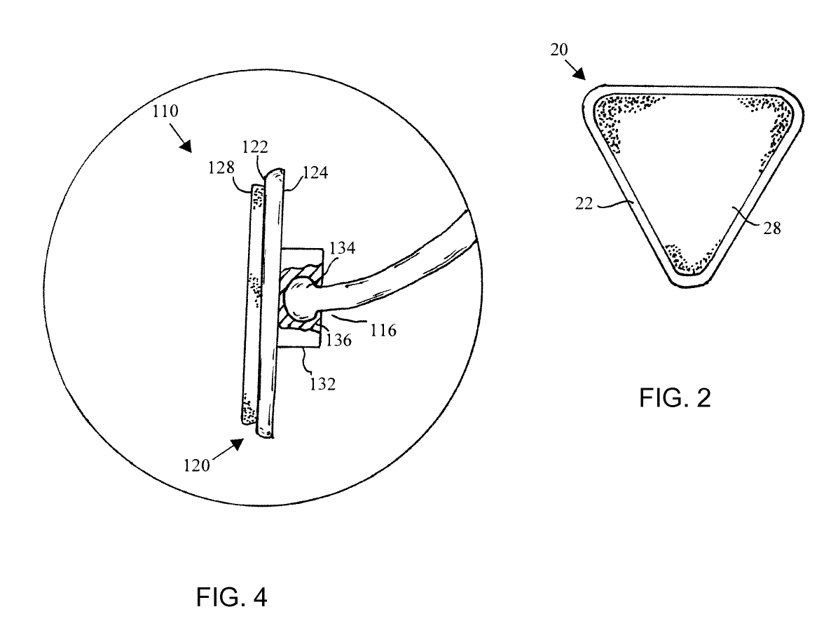

FIG. 2 illustrates the head portion 20, along line 2-2 in FIG. 1, of the first embodiment. As shown, the first surface 22 of the head portion 20 has a pad 28 covering a substantial portion of the first surface 22 of the head portion 20. It should be appreciated that the term “substantial portion” is synonymous with terms such as nearly, very nearly, about, approximately, etc. or can represent a certain percentage of coverage. For example, the pad 28 could cover a substantial portion or 75% of the first surface 22 of the head portion 20. In another example, the pad 28 could cover a substantial portion or 95% of the first surface 22 of the head portion 20. The pad 28 can be adhesively secured to the head portion 20 prior to surgery, and removed prior to sanitizing the apparatus 10. Alternatively, the pad 28 can be integrated into an elastic head cover that is removably installed over the head portion 20.

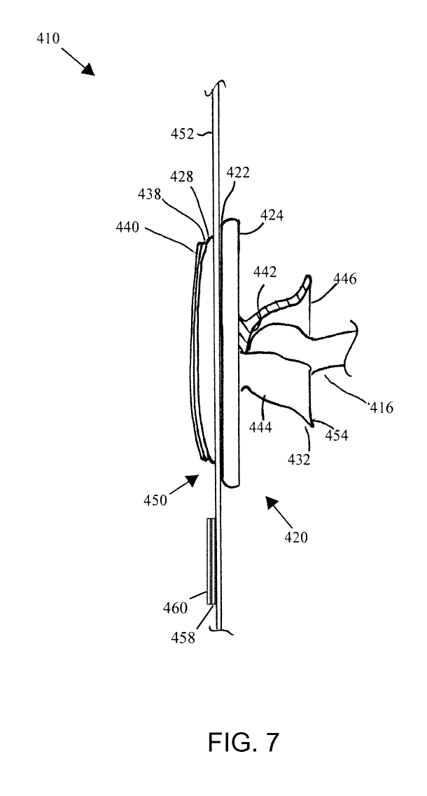

Also shown in FIG. 2 is that the head portion 20 in this embodiment has a generally triangular shape or configuration with rounded corners so as to fit properly into the perineum region of a patient. However, the head portion 20 can be any shape; for example, the shape of the head portion may be square, rectangular, oval, circular, or the like, and these or alternative configurations can be symmetrical or asymmetrical. Optionally, the pad 28 may include a convex outer surface (shown in FIG. 7) to provide accurate delivery of pressure to the bulbar membranous urethra with a minimum of difficulty and maximum safety and comfort for the patient.

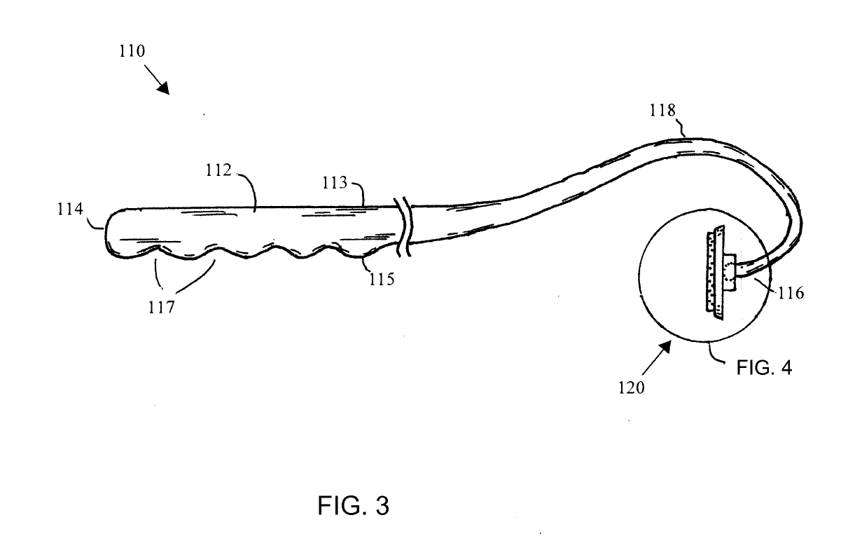

FIGS. 3 and 4 illustrate a second embodiment of the apparatus 110. The handle portion 112 is largely identical to the handle portion 12 on apparatus 10, except at its second end 116. In this embodiment the head portion 120 includes a receiving member 132 adapted pivotally to connect the head portion 120 to the second end 116 wherein the second end 116 is pivotally moveable within the receiving member 132. This pivot connection enables the handle portion 112 to be freely movable by the user while the head portion 120 of the apparatus maintains its position to facilitate consistent delivery of pressure against the perineum of a patient. However, once an ideal location by the user is established, the pivotal connection between the head portion 120 and the second end 116 may optionally include a locking mechanism, such as a threaded screw set, to prevent pivotal movement of the second end 116 within the receiving member 132.

As shown, the second end 116 includes a ball formation 134 matingly received in a socket 136 formed in the receiving member 132 where the second end 116 is pivotally moveable within the receiving member 132. The ball and socket configuration is preferably a snap fit connection, which allows the user to easily user to force to snap or press the second end 116 past the opening of the socket 136 and into and out of the cavity of the receiving member 132. In like manner, the receiving member 132 and the second end 116 are removably connected. In this embodiment, the apparatus 110 is preferably formed of a plastic material as described above. The ball and socket configuration can be configured to allow movement in a single plane (i.e., horizontal or vertical) or preferably for allowing free movement in any direction (i.e., 360 degrees).

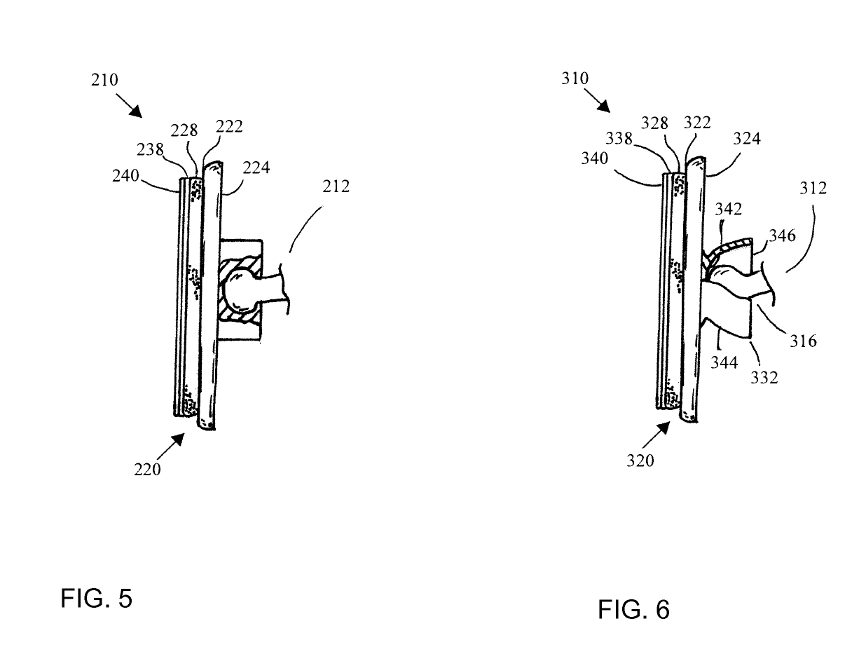

In a further embodiment shown in FIG. 5, the apparatus 210 is shown with its handle portion 212 broken away. The handle portion 212 is largely identical to handle portion 112, as described above. Here, the pad 228 further includes an adhesive layer 238 disposed on a side of the pad 228 opposite the first surface 222 of the head portion 220 and a release layer 240 removably attached to the adhesive layer 238. During use, the release layer 240 can be removed to allow the exposed adhesive layer 238 to be placed against the perineum area of the patient.

In another embodiment shown in FIG. 6, the apparatus 310 is shown with its handle portion 312 broken away. The handle portion 312 is largely identical to handle portion 112, as described above. Here, the first surface 322 of the head portion 320 includes a pad 328 with adhesive and release layers 338, 340 as described above. The second surface 324 of the head portion 320 includes as a receiving member 332 that is cup-shaped having a bottom surface 342 sized and configured to receive the second end 316, and a tapered continuous sidewall 344 that extends from the bottom surface to form an opening 346. In use, the enlarged opening 346 allows the user easily to engage the second end 316 with the cup-shaped receiving member 332, while also preventing unintended disengagement of the handle 312 and head portion 320. This configuration enables the user to localize the head portion 320 within the receiving member 332 without restricting the movement of the head portion 320 within the receiving member 332. Additionally, the extended wall portion 344 enables a safe range of movement of the apparatus 310 without interfering with the patient's genital area.

In another embodiment, the head portion 420 of the apparatus 410 of the present invention can also be integrated a surgical drapery system 450, as illustrated in FIG. 7. The surgical drapery system 450 may include a drapery layer 452, having at least one adhesive portion 458 and a release layer 460 removably attached to the adhesive portion 458. The surgical drapery system 450 may include one or more apertures to allow a patient's genitals to pass through the drapery layer 452; thus, allowing the adhesive portion 458 of the drapery layer 452 to be removably secured to the patients body. The surgical drapery system 450 may be made of one or more woven or nonwoven materials that are suitable for surgical drapery. In this embodiment, the cup-shaped receiving member 432 is shown to include an optional feature, which is a flared lip 454 that transitions from the tapered sidewall 444 to define the opening 446, as illustrated in FIG. 7. The flared lip portion 454 provide a larger target opening to help guide the second end 416 of the handle portion toward the bottom of the cup-shaped receiving member during the surgery. In this embodiment, the head portion 420 can be assembled by using a permanent adhesive to secure a perforated region of the drapery between the first surface 422 and the pad 428. It is contemplated that the cup-shaped receiving member 432 can be provided in multiple sizes and shapes to allow the apparatus to be utilized with retractors of different sizes and body types, such as for example, a handheld malleable retractor, a Richardson retractor, or the like. Alternatively, if desired, a user can grasp the cup-shaped receiving member 432 by hand to apply pressure to the perineum via the head portion.

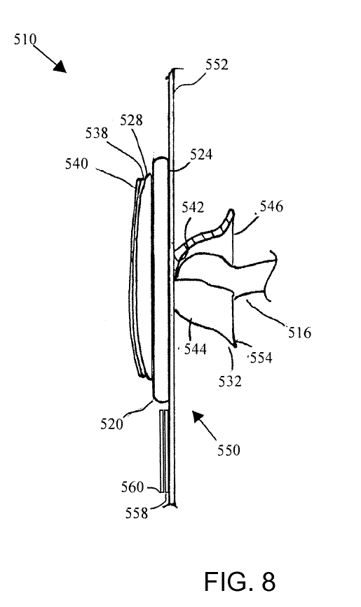

In a further embodiment shown in FIG. 8, the head portion 520 is also integrated with a surgical drapery system 550, similar to that illustrated in FIG. 7. The difference between the embodiments shown in FIGS. 7 and 8 concerns how the surgical drapery system 550 is connected to the head portion 520. Whereas in FIG. 7 the drapery 450 is sandwiched between the first surface 422 and the pad 428, in the embodiment shown in FIG. 8, the surgical drapery 550 is secured to the second surface 524 of the head portion 520 by adhesive or the like. In yet another embodiment, one side of the drapery can be secured adhesively to the pad and the opposite side can be provided with an adhesive layer/release layer to accommodate its direct, temporary attachment to the skin of the perineal region.

As with the embodiment shown in FIG. 7, it is contemplated that the cup-shaped receiving member 532 can be provided in multiple sizes and shapes to allow the apparatus to be utilized with retractors of different sizes and body types, such as for example, a handheld malleable retractor, a Richardson retractor, or the like. Alternatively, if desired, a user can grasp the cup-shaped receiving member 532 by hand to apply pressure to the perineum via the head portion. Usage of the cup-shaped receiving member 532 in this manner allows the same goal of applying perineal pressure, but without subscribing to a handle portion of a retractor.

As a further alternative to the embodiments shown in FIGS. 7 and 8, the cup-shaped receiving member 432, 532 may be substituted with a second pad to serve as a palpable marker for the location of the perineum that facilitates delivery of accurate pressure. In this instance, the second pad is intended to be used to deliver pressure manually rather than subscribing to a handle portion of a retractor.

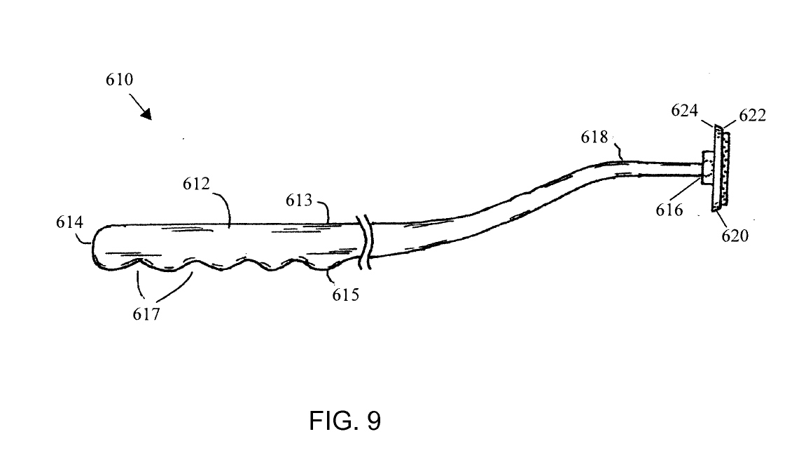

In another embodiment, FIG. 9 illustrates a further embodiment of the apparatus 610. The handle portion 612 is largely identical to the handle portion 12 on apparatus 10, except at its curvilinear segment 18 between the first and second end. In this embodiment, the curvilinear segment 618 includes an offset configuration between the first end 614 and the second end 616 such that the second end 616 faces in a direction opposite the first end and is positioned in a different plane in relation to the first end 614. In this embodiment, the configuration of the curvilinear segment 618 enables a user to grip and manipulate the apparatus 610 with a single hand while performing other tasks during the surgery with the free hand. In this embodiment, the head portion can be positioned to fit properly into the perineum region of a patient while an assistant positioned near a patient's lower extremities applies appropriate pressure to the perineum during an open prostatectomy.

Although the invention has been described in detail for the purposes of illustration, it is understood that such detail is solely for that purpose, and variations can be made therein by those skilled in the art without departing from the spirit and scope of the invention, which is defined by the following claims.

What is claimed:

1. A medical apparatus for perineal retraction during a prostatectomy, comprising:
a handle portion comprising a first end, a second end and a curvilinear segment between the first and second end; whereby the second end faces in a direction toward the first end; and
a head portion including a first surface on one side of the head portion and a second surface on an opposite side of the head portion, the second surface being connected to or engageable with the second end of the handle, and the head portion being sized and configured for placement against a perineum.

2. The apparatus according to claim 1, wherein the handle portion further comprises an upper side and a lower side opposite the upper side, and a plurality of spaced indentations disposed in the lower side.

3. The apparatus according to claim 1, wherein the head portion further comprises a pad covering a substantial portion of the first surface of the head portion.

4. The apparatus according to claim 3, wherein the pad comprises a gel or foam material.

5. The apparatus according to claim 3, wherein the pad comprises a convex outer surface.

6. The apparatus according to claim 1, wherein the head portion comprises a generally triangular configuration that is sized for placement against a perineum.

7. The apparatus according to claim 1, wherein the second surface of the head portion comprises a receiving member pivotally connected to the second end.

8. The apparatus according to claim 7, wherein the pivotal connection between the head portion and the second end includes a locking mechanism to prevent pivotal movement of the second end within a cavity of the receiving member.

9. The apparatus according to claim 7, wherein the receiving member and the second end are removably connected.

10. The apparatus according to claim 3, wherein the pad further comprises an adhesive layer disposed on a side of the pad opposite the first surface of the head portion and a release layer removably attached to the adhesive layer.

11. The apparatus according to claim 10, further comprising a surgical drapery layer fixed between the first surface of the head portion and the pad.

12. The apparatus according to claim 11, wherein the drapery layer includes at least one adhesive portion and a release layer removably attached to the adhesive portion located opposite the first surface of the head portion.

13. The apparatus according to claim 1, wherein the second surface is loosely engageable with the second end of the handle.

14. The apparatus according to claim 13, wherein the second surface of the head portion comprises a cup-shaped receiving member having an opening larger than the second end of the handle portion.

15. The apparatus according to claim 14, wherein the cup-shaped receiving member has a bottom surface sized and configured to receive the second end of the handle.

16. The apparatus according to claim 15, wherein the cup-shaped receiving member comprises a tapered sidewall that extends from the opening to the bottom surface.

17. The apparatus according to claim 16, wherein the cup-shaped receiving member further comprises flared lip portion that transitions the tapered sidewall to define the opening.

18. The apparatus according to claim 1, wherein the curvilinear segment has a gooseneck configuration.

19. The apparatus according to claim 1, wherein the curvilinear segment has a offset configuration, such that the second end is positioned in a different plane in relation to the first end and whereby the second end faces in a direction opposite the first end.

20. A medical apparatus for perineal retraction during a prostatectomy, comprising:
a head portion including a first surface on one side of the head portion and a second surface on an opposite side of the head portion, the head portion being sized and configured for placement against a perineum;
a pad covering a substantial portion of the first surface of the head portion; and
a surgical drapery layer secured between the first surface of the head portion and the pad or secured to the second surface of the head portion.

21. The apparatus according to claim 20, wherein the drapery layer includes at least one adhesive portion and a release layer removably attached to the adhesive portion located on the first surface on one side of the surgical drapery layer.

22. The apparatus according to claim 20, wherein the head portion comprises a generally triangular configuration that is sized for placement against a perineum.

23. The apparatus according to claim 20, wherein the pad further comprises an adhesive layer disposed on a side of the pad opposite the first surface of the head portion and a release layer removably attached to the adhesive layer.

24. The apparatus according to claim 20, wherein the pad comprises a gel or foam material.

25. The apparatus according to claim 20, wherein the pad comprises a convex outer surface.

26. The apparatus according to claim 20, wherein the second surface of the head portion comprises a cup-shaped receiving member having an opening.

27. A medical apparatus for perineal retraction during a prostatectomy, comprising:
a head portion including a first surface on one side of the head portion and a second surface on an opposite side of the head portion, the head portion being sized and configured for placement with the first surface adjacent a perineum; and
a surgical drapery layer secured to either the first surface or the second surface of the head portion.

28. The apparatus according to claim 27, wherein the head portion further comprises a cup-shaped receiving member formed on the second surface thereof, the cup-shaped receiving member having an opening and a bottom surface sized and configured to receive a handle.

29. The apparatus according to claim 28, further comprising a handle portion comprising an operable end that is sized and configure to mate with the bottom surface of the cup-shaped receiving member.

30. A method of performing a prostatectomy comprising:
applying the head portion of an apparatus according to claim 1 against the perineum of a patient, and retracting the perineum upon manipulation of the head portion.

31. A method of performing a prostatectomy comprising:
applying the head portion of an apparatus according to claim 20 against the perineum of a patient, and retracting the perineum upon manipulation of the head portion.

32. A method of performing a prostatectomy comprising:
applying the head portion of an apparatus according to claim 27 against the perineum of a patient, and retracting the perineum upon manipulation of the head portion.
